(19) United States
(12) Patent Application Publication
(10) Pub. No.: US 2004/0034106 A1
(43) Pub. Date: Feb. 19, 2004

(54) Treatment of anxiety disorders

(76) Inventors: Holly Ann Read, Indianapolis, IN (US); David Michelson, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/416,294

(22) PCT Filed: Nov. 6, 2001
(86) PCT No.: PCT/US01/27801

(51) Int. Cl. A61K 31/137

(52) U.S. Cl. 514/651

(57) ABSTRACT

Selective norepinephrine reuptake inhibitors are used to treat anxiety disorders, especially obsessive-compulsive disorder.

The invention belongs to the fields of pharmaceutical chemistry and central nervous system medicine, and provides a method of treatment for anxiety disorders.

Anxiety disorders represent the most prevalent type of psychiatric disorders in the United States. Anxiety disorders include panic disorder, obsessive-compulsive disorder, post-traumatic stress disorder, specific phobia, social phobia, and generalized anxiety disorder. All are characterized by uneasiness, a sense of fearfulness, and distress for no apparent reason. These disorders, if left untreated, reduce the quality of life and productivity of patients suffering from them. In the United States alone, more than 23 million people suffer from anxiety disorders. The cost to society from these disorders is staggering, estimated in 1990 at $46.6 billion in the United States alone in direct and indirect costs.

Currently available methods for treating anxiety disorders include behavioral therapy, cognitive therapy, and relaxation techniques. These methods typically take a considerable amount of time to achieve their desired effect. To increase the rate of recovery, these methods may be used in combination with one of a number of medications. Currently used medications include benzodiazepines, beta-blockers, buspirone, monoamine oxidase inhibitors, serotonin reuptake inhibitors, and tricyclic antidepressants, all of which have liabilities associated with their use. The benzodiazepines are potentially habit forming and can cause drowsiness; beta-blockers cannot be used if the patient has certain pre-existing medical conditions such as asthma, congestive heart failure, diabetes, vascular disease, hyperthyroidism, or angina pectoris; buspirone has a long induction period before its beneficial effects are realized; patients taking monoamine oxidase inhibitors are under strict dietary constraints and there is the potential for drug interactions, low blood pressure, moderate weight gain, reduced sexual response, and insomnia; the serotonin reuptake inhibitors can cause nausea, nervousness, and delayed ejaculation; and the tricyclic antidepressants can cause dry mouth, constipation, blurry vision, difficulty in urination, dizziness, low blood pressure, and moderate weight gain. New methods for treating anxiety disorders are needed which avoid or diminish the liabilities of current therapies.

The present invention provides a method for the treatment of anxiety disorders which comprises administering to a mammal in need of such treatment an effective amount of a selective norepinephrine reuptake inhibitor.

The present invention also provides a method for the prevention of anxiety disorders which comprises administering to a mammal susceptible to said disorders an effective amount of a selective norepinephrine reuptake inhibitor.

The present invention provides a method for the treatment or prevention of anxiety disorders that relies on a novel mechanism of action. This method comprises treating a mammal suffering from or susceptible to anxiety disorders with a compound that is a selective norepinephrine reuptake inhibitor. This mechanism is operative in mammals and the preferred mammal is a human.

A further embodiment of this invention comprises the administration of a composition that exhibits selective norepinephrine reuptake inhibitor activity. The composition may be composed of one or more agents that, individually or together, are selective inhibitors of norepinephrine reuptake.

The present invention also provides the use of a selective norepinephrine reuptake inhibitor for the preparation of a medicament useful for the treatment or prevention of anxiety disorders.

The present invention further provides the use of a selective norepinephrine reuptake inhibitor for the preparation of a medicament useful for the treatment of anxiety disorders with comorbid Attention-deficit Hyperactivity Disorder.

Many compounds, including those discussed at length below, are selective norepinephrine reuptake inhibitors, and no doubt many more will be identified in the future. In the practice of the present invention, it is intended to include reuptake inhibitors which show 50% effective concentrations of about 1000 nM or less, in the protocol described by Wong et al., Drug Development Research, 6, 397 (1985). The norepinephrine reuptake inhibitors useful for the method of the present invention are characterized in being selective for the inhibition of neurotransmitter reuptake relative to their ability to act as direct agonists or antagonists at other receptors. Norepinephrine reuptake inhibitors useful for the method of the present invention include, but are not limited to:

Tomoxetine, (R)-(−)-N-methyl-3-(2-methylphenoxy)-3-phenylpropylamine, is usually administered as the hydrochloride salt. Tomoxetine was first disclosed in U.S. Pat. No. 4,314,081. The word “tomoxetine” will be used here to refer to any acid addition salt or the free base of the molecule. See, for example, Gehlert, et al., Neuroscience Letters, 157, 203-206 (1993), for a discussion of tomoxetine's activity as a norepinephrine reuptake inhibitor; The compounds of formula I:

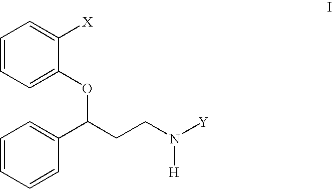

wherein X is C₁-C₄ alkylthio, and Y is C₁-C₂ alkyl or a pharmaceutically acceptable salt thereof. The compounds of formula I were described in U.S. Pat. No. 5,281,624, of Gehlert, Robertson, and Wong, and in Gehlert, et al., Life Sciences, 55(22), 1915-1920, (1995). The compounds are there taught to be inhibitors of norepinephrine reuptake in the brain. It is also explained that the compounds exist as stereoisomers, and that they accordingly include not only the racemates, but also the isolated individual isomers as well as mixtures of the individual isomers. For example, is the compounds of formula I include the following exemplary species:

N-ethyl-3-phenyl-3-(2-methylthiophenoxy)propylamine benzoate;

(R)-N-methyl-3-phenyl-3-(2-propylthiophenoxy)-propylamine hydrochloride;

(S)-N-ethyl-3-phenyl-3-(2-butylthiophenoxy)propylamine;

N-methyl-3-phenyl-3-(2-ethylthiophenoxy)propylamine malonate;

(S)-N-methyl-3-phenyl-3-(2-tert-butylthiophenoxy)-propylamine naphthalene-2-sulfonate;

(R)-N-methyl-3-(2-methylthiophenoxy)-3-phenyl-propylamine; and

Reboxetine (Edronax™), 2-[α-(2-ethoxy)phenoxy-benzyl]morpholine, is usually administered as the racemate. It was first taught by U.S. Pat. No. 4,229,449, which describes its utility for the treatment of depression. Reboxetine is a selective norepinephrine reuptake inhibitor. The term “reboxetine” will be used here to refer to any acid addition salt or the free base of the molecule existing as the racemate or either enantiomer.

While all compounds exhibiting norepinephrine reuptake inhibition are useful for the methods of the present invention, certain are preferred. It is preferred that the norepinephrine reuptake inhibitor is selective for norepinephrine over other neurotransmitters. It is especially preferred that the norepinephrine reuptake inhibitor be selected from tomoxetine, reboxetine, or (R)-N-methyl-3-(2-methylthiophenoxy)-3-phenylpropylamine. The use of tomoxetine hydrochloride for the methods of the present invention is the most preferred embodiment of the present invention.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

Many of the compounds used in this invention are amines, and accordingly react with any of a number of inorganic and organic acids to form pharmaceutically acceptable acid addition salts. Since some of the free amines of the compounds of this invention are typically oils at room temperature, it is preferable to convert the free mines to their pharmaceutically acceptable acid addition salts for ease of handling and administration, since the latter are routinely solid at room temperature. Acids commonly employed to form such salts are inorganic acids such as hydrochloric acid, hydrobromic acid, hydroiodic acid, sulfuric acid, phosphoric acid, and the like, and organic acids, such as p-toluenesultonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, b-hydroxybutyrate, glycollate, tartrate, methanesulfonate, propanesulfonate, naphthalene-1-sulfonate, naphthalene-2-sulfonate, mandelate and the like. Preferred pharmaceutically acceptable salts are those formed with hydrochloric acid.

The dosages of the drugs used in the present invention must, in the final analysis, be set by the physician in charge of the case using knowledge of the drugs, the properties of the drugs in combination as determined in clinical trials, and the characteristics of the patient including diseases other than that for which the physician is treating the patient. General outlines of the dosages, and some preferred dosages, can and will be provided here.

Tomoxetine: from about 5 mg/day to about 200 mg/day; preferably in the range from about 60 to about 150 mg/day; more preferably from about 60 to about 130 mg/day; and still more preferably from about 60 to about 120 mg/day;

Compounds of formula I: from about 0.01 mg/kg to about 20 mg/kg; preferred daily doses will be from about 0.05 mg/kg to 10 mg/kg; ideally from about 0.1 mg/kg to about 5 mg/kg;

Reboxetine: from about 1 to about 30 mg, once to four times/day; preferred, from about 5 to about 30 mg once/day.

All of the compounds concerned are orally available and are normally administered orally, and so oral administration is preferred. However, oral administration is not the only route or even the only preferred route. For example, transdermal administration may be very desirable for patients who are forgetful or petulant about taking oral medicine. Compounds of Formula I may also be administered by the percutaneous, intravenous, intramuscular, intranasal or intrarectal route, in particular circumstances. The route of administration may be varied in any way, limited by the physical properties of the drugs, the convenience of the patient and the caregiver, and other relevant circumstances (Remington's Pharmaceutical Sciences, 18th Edition, Mack Publishing Co. (1990)).

The pharmaceutical compositions are prepared in a manner well known in the pharmaceutical art. The carrier or excipient may be a solid, semi-solid, or liquid material that can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solutions, suspensions, or the like.

The compounds of the present invention may be administered orally, for example, with an inert diluent or capsules or compressed into tablets. For the purpose of oral therapeutic administration, the compounds may be incorporated with excipients and used in the form of tablets, troches, capsules, elixirs, suspensions, syrups, wafers, chewing gums and the like. These preparations should contain at least 4% of the compound of the present invention, the active ingredient, but may be varied depending upon the particular form and may conveniently be between 4% to about 70% of the weight of the unit. The amount of the compound present in compositions is such that a suitable dosage will be obtained. Preferred compositions and preparations according to the present invention may be determined by a person skilled in the art.

The tablets, pills, capsules, troches, and the like may also contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, as coatings. Thus, tablets or pills may be coated with sugar, shellac, or other coating agents. A syrup may contain, in addition to the present compounds, sucrose as a sweetening agent and certain preservatives, dyes and colorings and flavors. Materials used in preparing these various compositions should be pharmaceutically pure and non-toxic in the amounts used.

A formulation useful for the administration of R-(−)-N-methyl 3-((2-methylphenyl)oxy)-3-phenyl-1-aminopropane hydrochloride (tomoxetine) comprises a dry mixture of R-(−)-N-methyl 3-((2-methylphenyl)oxy)-3-phenyl-1-aminopropane hydrochloride with a diluent and lubricant. A starch, such as pregelatinized corn starch, is a suitable diluent and a silicone oil, such as dimethicone, a suitable lubricant for use in hard gelatin capsules. Suitable formulations are prepared containing about 0.4 to 26% R-(−)-N-methyl 3-((2-methylphen-yl)oxy)-3-phenyl-1-aminopropane hydrochloride, about 73 to 99% starch, and about 0.2 to 1.0% silicone oil. The following tables illustrate particularly preferred formulations:

| | 2.5 | 5 | 10 | 18 | 20 | 25 | 40 | 60 |
|---|---|---|---|---|---|---|---|---|
| | mg | mg | mg | mg | mg | mg | mg | mg |
| Ingredient (%) | | | | | | | | |
| R-(-)-N-methyl 3- | 1.24 | 2.48 | 4.97 | 8.94 | 9.93 | 12.42 | 19.87 | 22.12 |
| ((2-meth- | | | | | | | | |
| ylphenyl)oxy)-3- | | | | | | | | |
| phenyl-1- | | | | | | | | |
| aminopropane | | | | | | | | |
| hydrochloride | | | | | | | | |
| Dimethicone | 0.5 | 0.5 | 0.5 | 0.5 | 0.5 | 0.5 | 0.5 | 0.5 |
| Pregelatinized | 98.26 | 97.03 | 94.53 | 90.56 | 89.57 | 87.08 | 79.63 | 77.38 |
| Starch | | | | | | | | |
| Ingredient | | | | | | | | |
| (mg/capsule) | | | | | | | | |
| R-(-)-N-methyl 3- | 2.86 | 5.71 | 11.43 | 20.57 | 22.85 | 28.57 | 45.71 | 68.56 |
| ((2-meth- | | | | | | | | |
| ylphenyl)oxy)-3- | | | | | | | | |
| phenyl-1- | | | | | | | | |
| aminopropane | | | | | | | | |
| hydrochloride | | | | | | | | |
| Dimethicone | 1.15 | 1.15 | 1.15 | 1.15 | 1.15 | 1.15 | 1.15 | 1.55 |
| Pregelatinized | 225.99 | 223.14 | 217.42 | 208.28 | 206.00 | 200.28 | 183.14 | 239.89 |
| Starch | | | | | | | | |
| Capsule Fill | 230 | 230 | 230 | 230 | 230 | 230 | 230 | 310 |
| Weight (mg) | | | | | | | | |
| Capsule Size | 3 | 3 | 3 | 3 | 3 | 3 | 3 | 2 |

For the purpose of parenteral therapeutic administration, the compounds of the present invention may be incorporated into a solution or suspension. These preparations typically contain at least 0.1% of a compound of the invention, but may be varied to be between 0.1 and about 90% of the weight thereof. The amount of the compound of formula I present in such compositions is such that a suitable dosage will be obtained. The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Preferred compositions and preparations are able to be determined by one skilled in the art.

The compounds of the present invention may also be administered topically, and when done so the carrier may suitably comprise a solution, ointment, or gel base. The base, for example, may comprise one or more of the following: petrolatum, lanolin, polyethylene glycols, bees wax, mineral oil, diluents such as water and alcohol, and emulsifiers, and stabilizers. Topical formulations may contain a concentration of the formula I, or its pharmaceutical salt, from about 0.1 to about 10% w/v (weight per unit volume).

Inhibition or Norepinephrine Reuptake

The ability of compounds to inhibit the reuptake of norepinephrine may be measured by the general procedure of Wong, et al., supra.

Male Sprague-Dawley rats weighing 150-250 gm are decapitated and brains are immediately removed. Cerebral cortices are homogenized in 9 volumes of a medium containing 0.32 M sucrose and 10 mM glucose. Crude synaptosomal preparations are isolated after differential centrifugation at 1000×g for 10 minutes and 17,000×g for 28 minutes. The final pellets are suspended in the same medium and kept in ice until use within the same day.

Synaptosomal uptake of ³H-norepinephrine is determined as follows. Cortical synaptosomes (equvalent to 1 mg of protein) are incubated at 37° C. for 5 minutes in 1 mL Krebs-bicarbonate medium containing also 10 mM glucose, 0.1 mM iproniazide, 1 mM ascorbic acid, 0.17 mM EDTA and 50 nM ³H-norepinephrine. The reaction mixture is immediately diluted with 2 mL of ice-chilled Krebs-bicarbonate buffer and filtered under vacuum with a cell harvester (Brandel, Gaithersburg, Md.). Filters are rinsed twice with approximately 5 mL of ice-chilled 0.9% saline and the uptake of ³H-norepinephrine assessed by liquid scintillation counting. Accumulation of ³H-norepinephrine at 4° C. is considered to be background and is subtracted from all measurements. The concentration of the test compound required to inhibit 50% of the ³H-norepinephrine accumulation (IC₅₀ values) are determined by linear regression analysis.

Anxiety disorders are a heterogeneous class of diseases. The most common types of anxiety disorders are described in the following paragraphs.

Panic Disorder

Panic disorder is characterized by the sudden onset of intense apprehension, fearfulness, or terror. An attack of panic disorder is unprovoked and may last for a discrete period of time. During these attacks, it is not uncommon for the victim to experience shortness of breath, palpitations, chest pain or discomfort, choking or a smothering sensation, and fear of losing control.

Generalized Anxiety Disorder

Generalized anxiety disorder is characterized by at least 6 months of persistent and excessive anxiety and worry. It is associated with physical anxiety symptoms such as muscle aches, fatigue, difficulty sleeping, sweating, dizziness, and nausea.

Specific Phobia

Specific phobia is a persistent, intense, and irrational fear associated with a particular object or situation that leads to avoidance of that object or situation.

Social Phobia

Social phobia is a persistent fear of one or more situations in which the person is exposed to possible scutiny by others and the person fears that he or she may do something or act in a way that will be humiliating. Social phobias can include extreme shyness.

Obsessive-Compulsive Disorder

Obsessive-compulsive disorder is characterized by obsessions that cause anxiety and compulsions which serve to neutralize the anxiety. Common obsessions include fear of dirt, germs, or contamination or fear of harming someone; common compulsions are excessive cleaning, counting, double-checking, and hoarding.

Post-Traumatic Stress Disorder

Post-traumatic stress disorder is characterized by the re-experiencing of an extremely traumatic event accompanied by symptoms of increased arousal and by avoidance of the stimuli associated with the trauma. Individuals can become so preoccupied with the experience that they are unable to lead a normal life.

The diseases described above as well as other anxiety disorders contemplated by the method of the present invention are classified in the Diagnostic and Statistical Manual of Mental Disorders, 4th Version, published by the American Psychiatric Association (DSM). In such cases, the DSM code numbers are supplied below for the convenience of the reader.

| | |
|---|---|
| Panic Disorder Without Agoraphobia | DSM 300.01 |
| Panic Disorder With Agoraphobia | DSM 300.21 |
| Agoraphobia Without History of Panic Disorder | DSM 300.22 |
| Specific Phobia | DSM 300.29 |
| Social Phobia | DSM 300.23 |
| Obsessive-Compulsive Disorder | DSM 300.3 |
| Post-Traumatic Stress Disorder | DSM 309.81 |
| Acute Stress Disorder | DSM 308.3 |
| Generalized Anxiety Disorder | DSM 300.02 |
| Anxiety Disorder Due to a General Medical Condition | DSM 293.84 |
| Substance Induced Anxiety Disorder Alcohol | DSM 291.89 |
| Amphetamine (or Amphetamine-Like Substance) | DSM 292.89 |
| Caffeine | DSM 292.89 |
| Cannabis | DSM 292.89 |
| Cocaine | DSM 292.89 |
| Hallucinogen | DSM 292.89 |
| Inhalant | DSM 292.89 |
| Phencyclidine (or Phencyclidine-Like Substance) | DSM 292.89 |
| Sedative, Hypnotic, or Anxiolytic | DSM 292.89 |
| Other [Unknown] Substance | DSM 292.89 |
| Anxiety Disorder Not Otherwise Specified | DSM 300.00 |
| Separation Anxiety Disorder | DSM 309.21 |
| Sexual Adversion Disorder | DSM 302.79 |

Any of these disorders, whether presenting alone or in combination in an individual mammal, may be treated or prevented by the method of the present invention. The treatment of Obsessive-Compulsive Disorder is a preferred embodiment of the present invention.

Patients suffering from anxiety disorders also commonly suffer concomitantly from Attention-deficit Hyperactivity Disorder. The patient will receive benefit from the use of norepinephrine reuptake inhibitors in the amelioration of the symptoms of anxiety disorders regardless of whether comorbid conditions are present. Furthermore, a patient suffering from anxiety disorders and Attention-deficit Hyperactivity Disorder will receive benefit in the amelioration of symptoms of both conditions through the method of the present invention. A further embodiment of the present invention, therefore, is a method of treating anxiety disorders with comorbid Attention-deficit Hyperactivity Disorder comprising administering to a patient in need of treatment of both anxiety disorders and Attention-deficit Hyperactivity Disorder an effective amount of a selective norepinephrine reuptake inhibitor.

The method of the present invention is effective in the treatment of patients who are children, adolescents or adults, and there is no significant difference in the symptoms or the details of the manner of treatment among patients of different ages. In general terms, however, for purposes of the present invention, a child is considered to be a patient below the age of puberty, an adolescent is considered to be a patient from the age of puberty up to about 18 years of age, and an adult is considered to be a patient of 18 years or older.

### EXAMPLE 1

A female subject presented with chronic fingernail biting. The subject was treated with 60 mg of tomoxetine hydrochloride, twice daily for 13 consecutive days. At the time of final assessment the subject demonstrated significant improvement, with healthy appearing fingernails except for one finger. The patient's chronic fingernail biting behavior resumed upon termination of treatment with tomoxetine hydrochloride.

We claim:

1. Use of a selective norepinephrine reuptake inhibitor for the manufacture of a medicament for the treatment of anxiety of disorders.

2. Use according to claim 1 wherein the selective norepinephrine reuptake inhibitor is selected from the group consisting of tomoxetine, reboxetine, and a compound of formula I: 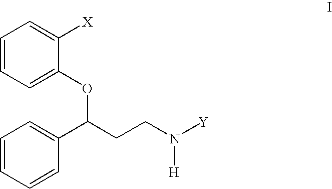
wherein X is C₁-C₄ alkylthio, and Y is C₁-C₂ alkyl or a pharmaceutically acceptable salt thereof.

3. Use according to claim 2 wherein the selective norepinephrine reuptake inhibitor is tomoxetine.

4. Use according to claim 2 wherein the selective norepinephrine reuptake inhibitor is tomoxetine hydrochloride.

5. Use according to any of claims 1-4 wherein Obsessive-Compulsive Disorder is treated.

6. Use of a selective norepinephrine reuptake inhibitor for the manufacture of a medicament for the treatment of anxiety disorders with comorbid Attention-deficit Hyperactivity Disorder.

7. Use according to claim 6 wherein the selective norepinephrine reuptake inhibitor is tomoxetine.

8. Use according to claim 7 wherein the selective norepinephrine reuptake inhibitor is tomoxetine hydrochloride.
